(19) United States
(12) Patent Application Publication
(10) Pub. No.: US 2024/0057970 A1
(43) Pub. Date: Feb. 22, 2024

(54) ULTRASOUND IMAGE ACQUISITION, TRACKING AND REVIEW

(71) Applicant: KONINKLIJKE PHILIPS N.V., EINDHOVEN (NL)

(72) Inventors: Shyam Bharat, Arlington, MA (US); Jochen Kruecker, Andover, MA (US); Claudia Errico, Medford, MA (US); Ramon Quido Erkamp, Swampscott, MA (US)

(21) Appl. No.: 18/269,050

(22) PCT Filed: Dec. 16, 2021
(86) PCT No.: PCT/EP2021/086045

§ 371 Date: Jun. 22, 2023

Related U.S. Application Data
(60) Provisional application No. 63/131,935, filed on Dec. 30, 2020.

(51) Int. Cl. A61B 8/00; A61B 8/08

(52) U.S. Cl. CPC A61B 8/4254; A61B 8/463; A61B 8/468; A61B 8/5207

(57) ABSTRACT

Systems and methods for ultrasound image acquisition, tracking and review are disclosed. The systems can include an ultrasound probe coupled with at least one tracking device configured to determine a position of the probe based on a combination of ultrasound image data and probe orientation data. The image data can be used to determine a physical reference point and superior-inferior probe coordinates within a patient being imaged, which can be supplemented with the probe orientation data to determine lateral coordinates of the probe. A graphical user interface can display imaging zones corresponding to a scan protocol, along with an imaging status of each zone based at least in part on the probe position. Ultrasound images acquired by the systems can be tagged with spatial indicators and severity indicators, after which the images can be stored for later retrieval and expert review.

### TECHNICAL FIELD

This application relates to systems configured to track the movement of an ultrasound probe and guide a user through various image acquisition protocols accordingly. More specifically, this application relates to systems and methods for acquiring and processing a combination of ultrasound image data and probe orientation data to track the position of an ultrasound probe and align the tracked position with image zones specific to a particular ultrasound scan protocol.

### BACKGROUND

Critical ultrasound scans are often performed in hectic settings under demanding time constraints. For example, lung ultrasound scans are frequently performed in intensive care units (ICUs) under a time limit of 15 minutes or less. Inexperienced ultrasound operators are commonly relied upon to perform such high-pressure scans, sometimes after only a few hours of formal training. As a result, faulty examinations plagued by low-quality and missing images are often utilized to arrive at incorrect patient diagnoses. Expert review of the ultrasound results, which may be performed remotely, can catch a portion of the acquisition mistakes, but such review is frequently unavailable or delayed due to staff shortages, thereby exacerbating the problem of inaccurate ultrasound-based diagnoses. Improved ultrasound systems configured to ensure the acquisition of complete, high-quality images necessary for various medical examinations are needed.

### SUMMARY

Ultrasound systems and methods for enhanced image acquisition, visualization and storage are disclosed. Embodiments involve determining and tracking the position of an ultrasound probe relative to a subject during an ultrasound examination. Real-time probe position tracking can be paired with acquisition guidance to ensure that no required images are missed during an examination. To facilitate accurate review of the acquired images, for example by an expert clinician not present during the examination, embodiments also involve tagging the images in their proper anatomical context and storing the tagged images for later retrieval.

In accordance with at least one example disclosed herein, an ultrasound imaging system may include an ultrasound probe configured to transmit ultrasound signals at a target region and receive echoes responsive to the ultrasound signals and generate radio frequency (RF) data corresponding to the echoes. The system may also include one or more image generation processors configured to generate image data from the RF data, along with an inertial measurement unit sensor configured to determine an orientation of the ultrasound probe. The system may also include a probe tracking processor configured to determine a current position of the ultrasound probe relative to the target region based on the image data and the orientation of the probe. The system may also include a user interface configured to display a live ultrasound image based on the image data. The user interface can also be configured to display one or more imaging zone graphics overlaid on a target region graphic, and the imaging zone graphics can correspond to a scan protocol. The user interface can also be configured to display an imaging status of each imaging zone represented by the imaging zone graphics.

In some embodiments, the ultrasound imaging system further includes a graphics processor configured to associate the current position of the ultrasound probe with one of the imaging zone graphics. In some embodiments, the imaging status indicates whether each imaging zone represented by one of the imaging zone graphics has been imaged, is currently being imaged, or has yet to be imaged. In some embodiments, the user interface is further configured to receive a user input tagging at least one of the imaging zone graphics with a severity level. In some embodiments, the ultrasound imaging system also includes a memory communicatively coupled to the user interface and configured to store at least one ultrasound image corresponding to each of the imaging zones. In some embodiments, the imaging status of each imaging zone is based on the current position of the ultrasound probe, a previous position of the ultrasound probe, a time spent by the probe at the current position and the previous position, a number of ultrasound images obtained at the current position and the previous position, or a combination thereof. In some embodiments, the probe tracking processor is configured identify a reference point within the target region based on the image data. In some embodiments, the reference point comprises a rib number. In some embodiments, the probe tracking processor is configured to determine superior-inferior coordinates of the probe based on the reference point. In some embodiments, the probe tracking processor is further configured to determine lateral coordinates of the probe based on the orientation of the probe. In some embodiments, the user interface is further configured to receive a target region selection, a patient orientation, or both.

In accordance with at least one example disclosed herein, a method may involve transmitting ultrasound signals at a target region using an ultrasound probe, receiving echoes responsive to the ultrasound signals, and generating radio frequency (RF) data corresponding to the echoes. The method may further involve generating image data from the RF data, determining an orientation of the ultrasound probe, and determining a current position of the ultrasound probe relative to the target region based on the image data and the orientation of the ultrasound probe. The method may also involve displaying a live ultrasound image based on the image data, and displaying one or more imaging zone graphics on a target region graphic, where the one or more imaging zone graphics correspond to a scan protocol. The method may further involve displaying an imaging status of each imaging zone represented by the imaging zone graphics.

In some embodiments, the method further involves associating the current position of the ultrasound probe with one of the imaging zone graphics. In some embodiments, the imaging status indicates whether each imaging zone represented by one of the imaging zone graphics has been imaged, is currently being imaged, or has yet to be imaged. In some embodiments, the method also involves receiving a user input tagging at least one of the imaging zone graphics with a severity level.

In some embodiments, the method also involves storing at least one ultrasound image corresponding to each of the imaging zones. In some embodiments, storing at least one ultrasound image involves spatially tagging the at least one ultrasound image with the corresponding imaging zone. In some embodiments, the imaging status of each imaging zone can be based on the current position of the ultrasound probe, a previous position of the ultrasound probe, a time spent by the probe at the current position and the previous position, a number of ultrasound images obtained at the current position and the previous position, or a combination thereof.

In some embodiments, the method further involves identifying a reference point within the target region based on the image data, determining superior-inferior coordinates of the probe based on the reference point, and determining lateral coordinates of the probe based on the orientation of the probe.

Embodiments can include a non-transitory computer-readable medium comprising executable instructions, which when executed cause a processor of a disclosed ultrasound imaging system to perform any of the aforementioned methods.

### BRIEF DESCRIPTION OF THE DRAWINGS

FIG. 1 is a block diagram of an ultrasound imaging system arranged according to principles of the present disclosure.

FIG. 2 is a block diagram illustrating an example processor arranged according to principles of the present disclosure.

FIG. 3 is a graphical user interface displayed according to examples of the present disclosure.

FIG. 4 is a diagram showing aspects of post-acquisition image storage, retrieval and review implemented according to examples of the present disclosure.

FIG. 5 is a schematic of an ultrasound probe tracking technique implemented according to embodiments of the present disclosure.

FIG. 6 is a flow chart of an example process implemented according to embodiments of the present disclosure.

FIG. 7 is a flow chart of another example process implemented according to embodiments of the present disclosure.

### DESCRIPTION

The following description of certain examples is in no way intended to limit the disclosure or its applications or uses. In the following detailed description of examples of the present systems and methods, reference is made to the accompanying drawings which form a part hereof, and in which are shown by way of illustration specific examples in which the described systems and methods may be practiced. These examples are described in sufficient detail to enable those skilled in the art to practice the presently disclosed systems and methods, and it is to be understood that other examples may be utilized and that structural and logical changes may be made without departing from the spirit and scope of the present disclosure. Moreover, for the purpose of clarity, detailed descriptions of certain features will not be discussed when they would be apparent to those skilled in the art so as not to obscure the description of the present disclosure. The following detailed description is therefore not to be taken in a limiting sense, and the scope of the present systems and methods is defined only by the appended claims.

Ultrasound systems configured to provide real-time probe tracking and guidance are disclosed, along with associated methods of displaying, tagging and archiving acquired images for subsequent review. In some examples, a graphical user interface can be configured to display one or more image zones relevant to a particular scan protocol, such as a lung scan. The image zones can be depicted in the form of dynamic graphics overlaid on a patient rendering or live ultrasound image. By tracking the ultrasound probe position during a scan, the systems disclosed herein can also update the status of each image zone depicted on the user interface in real time to reflect whether the zone has already been imaged, is currently being imaged, or has yet to be imaged. In this manner, the user can be guided through a scan protocol until all necessary images (from all required zones) are obtained. The acquired images can be saved as they are acquired for later review, and each image can be spatially tagged with its corresponding image zone. In this manner, the acquired images can be stored in predefined image zone “buckets,” each bucket corresponding to a specific anatomical area of a patient, thereby allowing a post-acquisition reviewer to examine the images in their proper anatomical context. For example, post-acquisition reviewers can analyze images from one or more zones of interest in systematic fashion without having to decipher which images correspond to which regions of the body.

While the present disclosure is not limited to any particular scan protocol or patient anatomy, embodiments disclosed herein are described in connection with lung scans for illustrative purposes only. Lung scans may be particularly amenable to improvement via the systems disclosed herein due to the visually and spatially diverse findings commonly associated with lung-related ailments, non-limiting examples of which may include COVID-19, pneumonia, lung cancer, or physical injury. Clinicians analyzing lung scan results are often forced to manually reconcile multiple streams of fragmented information in order to arrive at a final conclusion or diagnosis, a task that is often difficult to accomplish as less-experienced staff are increasingly relied upon to perform lung scans. Moreover, images that are incorrectly annotated and/or tagged by the user performing the scan make it difficult to link the images to their corresponding anatomical locations, which also complicates longitudinal studies and monitoring of various lung conditions. As noted above, the disclosed systems and methods are not limited to evaluations of the lungs, and may be readily applied to a subject's heart, legs, arms, etc. The disclosed embodiments are also not confined to human subjects, and may be applied to animals as well, for example pursuant to scan protocols performed in veterinary settings.

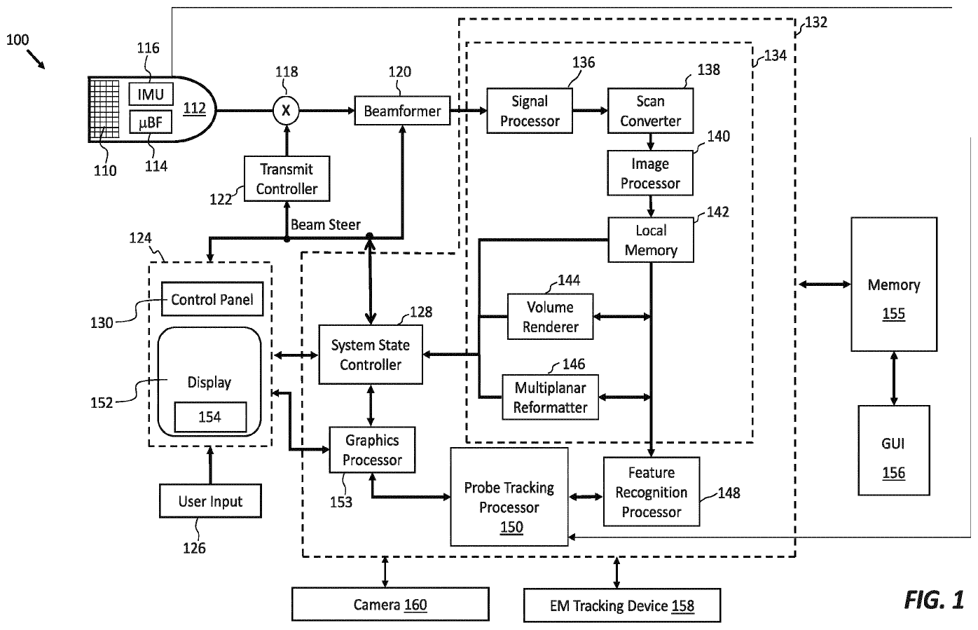

FIG. 1 shows a block diagram of an ultrasound imaging system 100, which may be mobile or cart-based, constructed in accordance with the principles of the present disclosure. Together, the components of the system 100 can acquire, process, display and store ultrasound image data corresponding to a subject, e.g., a patient, and determine which regions of the subject have been imaged, are currently being imaged, or have yet to be adequately imaged pursuant to a particular scan protocol.

As shown, the system 100 may include a transducer array 110, which may be included in an ultrasound probe 112, for example an external ultrasound probe. In other examples, the transducer array 110 may be in the form of a flexible array configured to be conformally applied to a surface of subject to be imaged (e.g., patient). The transducer array 110 is configured to transmit ultrasound signals (e.g., beams, waves) and receive echoes (e.g., received ultrasound signals) responsive to the transmitted ultrasound signals. A variety of transducer arrays may be used, e.g., linear arrays, curved arrays, or phased arrays. The transducer array 110, for example, can include a two dimensional array (as shown) of transducer elements capable of scanning in both elevation and azimuth dimensions for 2D and/or 3D imaging. As is generally known, the axial direction is the direction normal to the face of the array (in the case of a curved array the axial directions fan out), the azimuthal direction is defined generally by the longitudinal dimension of the array, and the elevation direction is transverse to the azimuthal direction.

In some examples, the transducer array 110 may be coupled to a microbeamformer 114, which may be located in the ultrasound probe 112, and which may control the transmission and reception of signals by the transducer elements in the array 110. In some examples, the microbeamformer 114 may control the transmission and reception of signals by active elements in the array 110 (e.g., an active subset of elements of the array that define the active aperture at any given time).

The ultrasound probe 112 can also include an inertial measurement unit sensor (IMU sensor) 116, which may comprise a gyroscope in some examples. The IMU sensor 116 can be configured to detect and measure the motion of the ultrasound probe 112, for example by determining its orientation, which can be utilized to determine its lateral/medial and anterior-posterior position relative to the subject being imaged.

In some examples, the microbeamformer 114 may be coupled, e.g., by a probe cable or wirelessly, to a transmit/receive (T/R) switch 118, which switches between transmission and reception and protects a main beamformer 120 from high-energy transmit signals. In some embodiments, for example in portable ultrasound systems, the T/R switch 118 and other elements in the system can be included in the ultrasound probe 112 rather than in the ultrasound system base, which may house the image processing electronics. An ultrasound system base typically includes software and hardware components including circuitry for signal processing and image data generation as well as executable instructions for providing a user interface.

The transmission of ultrasonic signals from the transducer array 110 under control of the microbeamformer 114 may be directed by a transmit controller 122, which can be coupled to the T/R switch 118 and the main beamformer 120. The transmit controller 122 may control characteristics of the ultrasound signal waveforms transmitted by the transducer array 110, for example, amplitude, phase, and/or polarity. The transmit controller 122 may also control the direction in which beams are steered. Beams may be steered straight ahead from (orthogonal to) the transducer array 110, or at different angles for a wider field of view. The transmit controller 122 may also be coupled to a graphical user interface (GUI) 124 configured to receive one or more user inputs 126. For example, the user may be the person performing the ultrasound scan and may select, via the GUI 124, whether the transmit controller 122 causes the transducer array 110 to operate in a harmonic imaging mode, fundamental imaging mode, Doppler imaging mode, or a combination of imaging modes (e.g., interleaving different imaging modes). User input 126 comprising one or more imaging parameters can be transmitted to a system state controller 128 communicatively coupled to the GUI 124, as further described below.

Additional examples of user input 126 can include a scan type selection (e.g., lung scan), a front or back side of the patient, a patient condition (e.g., pneumonia), and/or an estimated severity level of one more features or conditions captured in a particular ultrasound image. The user input 126 can also include various types of patient information, including but not limited a patient's name, age, height, body weight, medical history, etc. The date and time of the current scan may also be input, along with the name of the user performing the scan. To receive the user input 126, the GUI 124 may include one or more input devices such as a control panel 130, which can include one or more mechanical controls (e.g., buttons, encoders, etc.), touch-sensitive controls (e.g., a trackpad, a touchscreen, or the like), and/or other known input devices (e.g., voice command receivers) responsive to a variety of auditory and/or tactile inputs. Via the control panel 130, the GUI 124 may also be used to adjust various parameters of image acquisition, generation, and/or display. For example, a user may adjust the power, imaging mode, level of gain, dynamic range, turn on and off spatial compounding, and/or level of smoothing.

In some examples, the partially beamformed signals produced by the microbeamformer 114 may be coupled to the main beamformer 120, where partially beamformed signals from individual patches of transducer elements may be combined into a fully beamformed signal. The microbeamformer 114 can also be omitted in some examples, and the transducer array 110 may be under the control of the main beamformer 120, which can then perform all beamforming of signals. In examples with and without the microbeamformer 114, the beamformed signals of main beamformer 120 are coupled to image processing circuitry 132, which may include one or more image generation processors 134, examples of which can include a signal processor 136, a scan converter 138, an image processor 140, a local memory 142, a volume renderer 144, and/or a multiplanar reformatter 146. Together, the image generation processors 134 can be configured to produce live ultrasound images from the beamformed signals (e.g., beamformed RF data).

The signal processor 136 may receive and process the beamformed RF data in various ways, such as bandpass filtering, decimation, and I and Q component separation. The signal processor 136 may also perform additional signal enhancement such as speckle reduction, signal compounding, and electronic noise elimination. Output from the signal processor 136 may be coupled to the scan converter 138, which may arrange the echo signals in the spatial relationship from which they were received in a desired image format. For instance, the scan converter 138 may arrange the echo signals into a two dimensional (2D) sector-shaped format.

The image processor 140 is generally configured to generate image data from the RF data, and may perform additional enhancement such as contrast and intensity optimization. Radiofrequency data acquired by the ultrasound probe 112 can be processed into various types of image data, non-limiting examples of which may include per-channel data, pre-beamformed data, post-beamformed data, log-detected data, scan converted data, and processed echo data in 2D and/or 3D. Output (e.g., B-mode images) from the image processor 140 may be coupled to the local image memory 142 for buffering and/or temporary storage. The local memory 142 may be implemented as any suitable non-transitory computer readable medium (e.g., flash drive, disk drive), configured to store data generated by the system 100 including images, executable instructions, user inputs 126 provided by a user via the GUI 124, or any other information necessary for the operation of the system 100.

In embodiments configured to generate a clinically-relevant volumetric subset of image data, the volume renderer 144 can be included to generate an image (also referred to as a projection, render, or rendering) of the 3D dataset as viewed from a given reference point, e.g., as described in U.S. Pat. No. 6,530,885 (Entrekin et al.). The volume renderer 144 may be implemented as one or more processors in some examples. The volume renderer 144 may generate a render, such as a positive render or a negative render, by any known or future known technique such as surface rendering and maximum intensity rendering. The multiplanar reformatter 146 may convert echoes which are received from points in a common plane in a volumetric region of the body into an ultrasonic image of that plane, as described in U.S. Pat. No. 6,443,896 (Detmer).

In some examples, output from the image processor 140, local memory 142, volume renderer 144 and/or multiplanar reformatter 146 may be transmitted to a feature recognition processor 148 configured to recognize various anatomical features and/or image features within a set of image data. Anatomical features can include various organs, bones, bodily structures or portions thereof, while image features can include one or more image artifacts. Embodiments of the feature recognition processor 148 may be configured to recognize such features by referencing and sorting through a large library of stored images.

Image data received from one or more components of the image generation processors 134, and in some examples, the feature recognition processor 148, can then be received by a probe tracking processor 150. The probe tracking processor 150 can process the received image data together with the data output from the IMU sensor 116 to determine the position of the probe 112 relative to a subject being imaged. The probe tracking processor 150 can also measure the time the probe 112 spends at each position. As further set forth below, the probe tracking processor 150 may determine the probe position by using, as a reference point, one or more features captured in the ultrasound images and recognized by the feature recognition processor 148. The reference points gleaned from the image data are then augmented by probe orientation data received from the IMU sensor 116. Together, these inputs can be used to determine the position of the probe and the corresponding scan-specific zone being imaged.

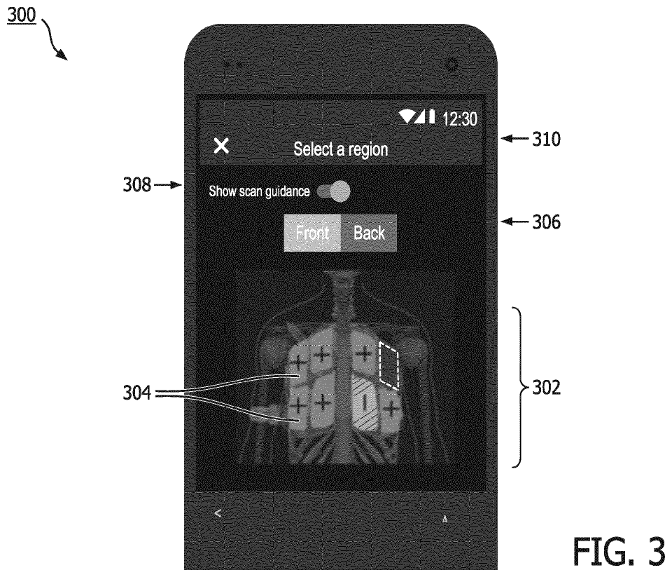

The system state controller 128 may generate graphic overlays for displaying on one or more displays 152 of the GUI 124. These graphic overlays can contain, for example, standard identifying information such as patient name, date and time of the image, imaging parameters, and the like. For these purposes, the system state controller 128 may be configured to receive input from the GUI 124, such as a typed patient name or other annotations. The graphic overlays can also portray discrete imaging zones specific to a particular scan protocol and/or patient condition, along with an imaging status of each zone. Graphic overlays of imaging zones can be displayed on a schematic depiction of at least a portion of the subject, as shown below in FIG. 3, or directly on a previously-acquired or live ultrasound image.

To display and update the status of each imaging zone graphic, embodiments may also include a graphics processor 153 communicatively coupled to the user interface 124, system state controller 128, and probe tracking processor 150. The graphics processor 153 can be configured to associate the current position of the ultrasound probe 112, as determined by the probe tracking processor 150, with one of the imaging zones and corresponding graphics depicted on the display 152 of the GUI 124, for example by transforming the physical probe coordinates determined by the probe tracking processor 150 into pixel regions of the display 152. Whether certain pixels corresponding to the probe coordinates fall within a particular imaging zone graphic can also be determined by the graphics processor 153. Relatedly, the graphics processor 153 may also be configured to determine and/or update the imaging status of each imaging zone based at least in part on one or more current and previous positions of the ultrasound probe 112 as determined by the probe tracking processor 150. For example, a “currently-imaging” zone graphic can be switched to a “previously-imaged” zone graphic based on a new position of the probe 112 determined by the probe tracking processor 150, which the graphics processor 153 can translate into an updated imaging status, either alone or with additional processing provided by the system state controller 128, the GUI 124, or both. The graphics processor 153 can also update the imaging status of each imaging zone graphic based on the time spent by the probe 112 at a given position or range of positions, along with the number of ultrasound images generated by the image generation processors 134 at the current probe position or range of positions. For example, if the probe 112 only acquires image data at a certain position or cluster of positions for a brief moment, e.g., five or ten seconds, the graphics processor 153 can maintain the “currently-imaging” or “yet-to-be imaged” status of the imaging zone graphic corresponding to the imaging zone encompassing that position or cluster of positions.

The display 152 may include a display device implemented using a variety of known display technologies, such as LCD, LED, OLED, or plasma display technology. In some examples, the display 152 may overlap with the control panel 130, such that a user can interact directly with the images shown on the display 152, for example by touch-selecting certain anatomical features for enhancement, indicating which image zones have been adequately imaged, assigning a severity level to one or more acquired images or corresponding zones, and/or selecting an anatomical orientation for image zone display. The display 152 can also show one or more ultrasound images 154, including a live ultrasound image and in some examples, a still, previously acquired image. In some examples, the display 152 may be a touch-sensitive display that includes one or more soft controls of the control panel 130.

As further shown, the system 100 can include or be communicatively coupled with an external memory 155, which may store various types of data, including raw image data, processed ultrasound images, patient-specific information, annotations, clinical notes, and/or image labels. The external memory 155 can store images tagged with image zone information, such as spatial tags for each image corresponding to the image zones from which they were acquired, and/or severity tags assigned to the images and/or zones they were acquired from. In this manner, the stored images are directly associated with a region of the subject, e.g., a lung or a portion of a lung, and flagged with an estimated severity level of a potential medical condition. The images stored in the external memory 155 can be referenced over time, thereby enabling longitudinal assessment of a subject and one or more features of interest identified therein. In some examples, the stored images can be used prospectively to tailor a scan protocol based on the clinical information embodied in the images. For instance, if only one imaging zone is of particular interest to a clinician, for example because a lesion is present within the portion of the body corresponding to that zone, and/or a moderate- to high-severity tag was assigned to that zone, then a user reviewing the stored images can use that information to focus future imaging efforts.

Embodiments described herein can also include at least one additional GUI 156 configured to display acquired images to a clinician, for example after an ultrasound scan has been completed. GUI 156 can be positioned in a different location than GUI 124, thereby allowing the clinician to analyze the acquired images remotely. The images retrieved and displayed on the GUI 156 can include images stored in the external memory 155, along with the spatial tags, severity tags, and/or other annotations and labels associated with the images.

As further shown, the system 100 may include or be coupled with one or additional or alternative devices configured to determine or refine the position of the ultrasound probe 112. For example, an electromagnetic (EM) tracking device 158 can be included. The EM tracking device 158 can comprise a tabletop field generator, which may be positioned under or behind the patient, depending on whether the patient is lying down or sitting. The system 100 can be calibrated by defining the boundaries of the targeted scanning area, which may be accomplished by tracking the ultrasound probe 112 as it is placed at the neck, abdomen, left side and right side of the patient. After calibration, the system 100 can be used to track the probe 112 spatially and temporally without the aid of the IMU sensor 116, while also mapping the area of the target region being scanned.

The system 100 can additionally or alternatively include a camera 160 mounted in the examination room containing the system 100, integrated into the probe 112, or otherwise coupled with the GUI 124. Images obtained using the camera can be used to estimate the current imaging zone being scanned, for example by recognizing the features present in the camera images. In some examples, the image data gathered by the camera 160 can be used to supplement the ultrasound image data and the data received from the IMU sensor 116 to further improve the accuracy of the probe tracking processor 150.

In some embodiments, various components shown in FIG. 1 may be combined. For instance, the feature recognition processor 148 and probe tracking processor 150 may be implemented as a single processor, as can the system state controller 128 and graphics processor 153. Various components shown in FIG. 1 may also be implemented as separate components. In some examples, one or more of the various processors shown in FIG. 1 may be implemented by general purpose processors and/or microprocessors configured to perform the specified tasks described herein. In some examples, one or more of the various processors may be implemented as application specific circuits. In some examples, one or more of the various processors (e.g., image processor 140) may be implemented with one or more graphical processing units (GPUs).

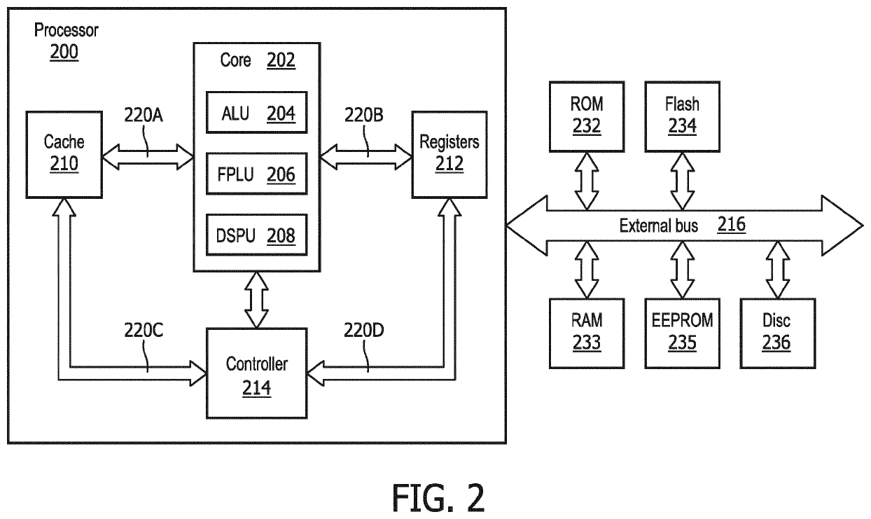

FIG. 2 is a block diagram illustrating an example processor 200 utilized according to principles of the present disclosure. Processor 200 may be used to implement one or more processors described herein, such as the image processor 140 shown in FIG. 1. Processor 200 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific circuit (ASIC) where the ASIC has been designed to form a processor, or a combination thereof.

The processor 200 may include one or more cores 202. The core 202 may include one or more arithmetic logic units (ALU) 204. In some examples, the core 202 may include a floating point logic unit (FPLU) 206 and/or a digital signal processing unit (DSPU) 208 in addition to or instead of the ALU 204.

The processor 200 may include one or more registers 212 communicatively coupled to the core 202. The registers 212 may be implemented using dedicated logic gate circuits (e.g., flip-flops) and/or any memory technology. In some examples the registers 212 may be implemented using static memory. The register may provide data, instructions and addresses to the core 202.

In some examples, processor 200 may include one or more levels of cache memory 210 communicatively coupled to the core 202. The cache memory 210 may provide computer-readable instructions to the core 202 for execution. The cache memory 210 may provide data for processing by the core 202. In some examples, the computer-readable instructions may have been provided to the cache memory 210 by a local memory, for example, local memory attached to the external bus 216. The cache memory 210 may be implemented with any suitable cache memory type, for example, metal-oxide semiconductor (MOS) memory such as static random access memory (SRAM), dynamic random access memory (DRAM), and/or any other suitable memory technology.

The processor 200 may include a controller 214, which may control input to the processor 200 from other processors and/or components included in a system (e.g., GUI 124) and/or outputs from the processor 200 to other processors and/or components included in the system (e.g., display 152). Controller 214 may control the data paths in the ALU 204, FPLU 206 and/or DSPU 208. Controller 214 may be implemented as one or more state machines, data paths and/or dedicated control logic. The gates of controller 214 may be implemented as standalone gates, FPGA, ASIC or any other suitable technology.

The registers 212 and the cache memory 210 may communicate with controller 214 and core 202 via internal connections 220A, 220B, 220C and 220D. Internal connections may implemented as a bus, multiplexor, crossbar switch, and/or any other suitable connection technology.

Inputs and outputs for the processor 200 may be provided via a bus 216, which may include one or more conductive lines. The bus 216 may be communicatively coupled to one or more components of processor 200, for example the controller 214, cache 210, and/or register 212. The bus 216 may be coupled to one or more components of the system, such as display 152 and control panel 130 mentioned previously.

The bus 216 may be coupled to one or more external memories. The external memories may include Read Only Memory (ROM) 232. ROM 232 may be a masked ROM, Electronically Programmable Read Only Memory (EPROM) or any other suitable technology. The external memory may include Random Access Memory (RAM) 233. RAM 233 may be a static RAM, battery backed up static RAM, Dynamic RAM (DRAM) or any other suitable technology. The external memory may include Electrically Erasable Programmable Read Only Memory (EEPROM) 235. The external memory may include Flash memory 234. The external memory may include a magnetic storage device such as disc 236. In some examples, the external memories may be included in a system, such as ultrasound imaging system 100 shown in FIG. 1, for example local memory 142.

FIG. 3 is an example of a graphical user interface (GUI) 300 configured to guide a user through an ultrasound scan by depicting each imaging zone relevant to that particular scan, along with the imaging status of each zone. The GUI 300 displays a patient graphic 302 depicting at least a portion of the patient's body. In this example, the patient graphic 302 depicts the patient's chest region. A plurality of discrete imaging zones 304 are depicted in the form of imaging zone graphics within the patient graphic 302, totaling eight zones in this example. The imaging status of each imaging zone 304 can be indicated by modifying the appearance of each zone as the scan is performed. For example, the color of each imaging zone 304 may be updated as a user acquires images therefrom. In one specific embodiment, imaging zones that have already been scanned may be colored green, while zones that have not been scanned can be shown in red, and the zone currently being scanned can be shown in orange. The particular colors representing each zone status can of course vary. As shown in FIG. 3, the imaging zone currently being imaged is labeled with parallel, diagonal lines, and the lone imaging zone that has not been imaged is labeled with a dashed line around its perimeter. The rest of the depicted imaging zones have already been imaged.

As further shown, the GUI 300 can also provide a symbol indicating whether sagittal and transverse images have been acquired from each imaging zone. In this particular example, the “+” sign indicates that both sagittal and transverse images have indeed been captured, while the “|” sign indicates that only sagittal images have been captured, and although not visible in this particular snapshot, a “−” sign can be shown to indicate that only transverse images have been acquired. The GUI 300 thus provides a comprehensive reference for the user to determine, in real time, whether any zones have been inadvertently missed and whether additional images are necessary.

The number of imaging zones 304 may vary depending on the scan protocol. For example, protocols may require obtaining at least one image from one zone, two zones, three zones, four zones, five zones, six zones, seven zones, eight zones, nine zones, ten zones, 11 zones, 12 zones, 13 zones, 14 zones, 15 zones, 16 zones, or more. To perform a comprehensive examination of the lungs, for instance, a multi-zone protocol may include about six, eight, 12 or 14 imaging zones. Protocols can also be customized according to certain embodiments, such that instead of performing a comprehensive scan of one or more organs or regions of a subject, a subset of zones may be specified for imaging. For example, a clinician may only designate one or two zones for imaging due to the abnormalities previously identified in the areas of the body represented by such zones. In this manner, the efficiency of longitudinal monitoring accomplished via ultrasound imaging can be improved.

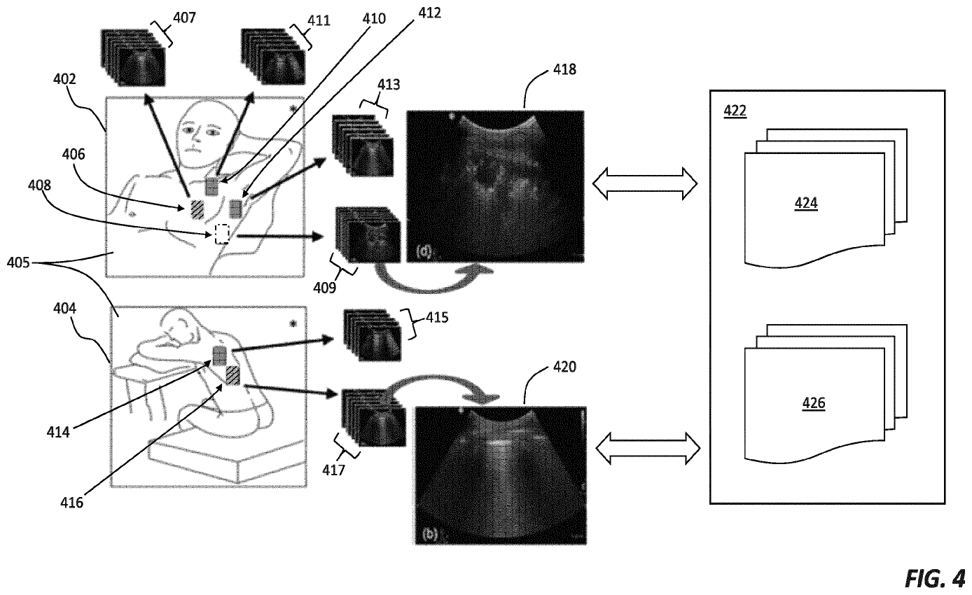

After an imaging zone 304 has been completely scanned, the user can be prompted to enter an estimated severity rating, for example a numerical rating on a scale ranging from 1 to 5, based on the observed anatomical and/or imaging features captured in that particular imaging zone. This real-time tagging of imaging zones and/or at least one image associated therewith, can be used to guide or prioritize post-acquisition review efforts, as further described below in connection with FIG. 4. In some embodiments, the systems disclosed herein (e.g., system 100) can be configured to automatically identify certain anatomical and/or imaging features embodied in the acquired image data. The feature recognition processor 148 shown in FIG. 1, for instance, can identify such features for display and/or to inform the probe tracking processor 150.

As further shown, the GUI 300 can include a patient orientation selection 306, which in this embodiment comprises a front/back selection. The patient orientation selection 306 can comprise a touch-sensitive control that allows a user to toggle between a front view and a back view of the subject being imaged, along with the imaging zones associated with each view. The displayed patient graphic 302 shows a front view divided into eight imaging zones 304. A back view may include the same or different number of imaging zones.

The GUI 300 also includes a scan guidance selection 308, here in the form of a touch-sensitive slide control, that allows a user to turn the scan guidance on and off. If the scan guidance is turned off, the imaging zones 304 and/or their corresponding imaging statuses may be removed from the patient graphic 302.

An anatomical region selection 310 can also be provided on the GUI 300 to allow the user to input an anatomical region for examination, which may cause the GUI 300 to display the imaging zones relevant to that particular region. Example regions can include a chest region or an anatomical feature therein, such as the heart or the lungs. The GUI 300 can be configured to receive the region selection 310 via free-text input manually by the user and/or via selection from a menu, e.g., dropdown menu.

FIG. 4 is a diagram of a post-acquisition storage and review scheme implemented in accordance with the systems and methods described herein. As shown, a front view 402 and a back view 404 of a subject, each including one or more imaging zones, can be displayed on a GUI 405 for review by a clinician during or after an ultrasound scan, for example at a remote location. GUI 405 may thus correspond to GUI 156 shown in FIG. 1. The imaging zone graphics may indicate an estimated severity level of a medical condition or abnormality within each zone, as perceived by the ultrasound operator during the scan.

The estimated severity levels can flag potential issues for later review. For example, the front view 402 includes a moderate zone 406, a severe zone 408, and two normal zones 410, 412. Images acquired from each zone can be spatially-tagged by one or more processors (e.g., probe tracking processor 150 and system state controller 128), such that the images are organized and stored in relevant zone storage buckets, each bucket corresponding to a specific imaging zone. In this example, a plurality of images 407 were acquired, organized and stored together in a discrete storage bucket corresponding to the moderate zone 406. A plurality of images 409 were acquired and stored a discrete storage bucket corresponding to the severe zone 408. A plurality of images 411 were archived in a storage bucket corresponding to one of the normal zones 410, and a separate plurality of images 413 have been archived for the other normal zone 412. For the back view 404, a normal zone 414 is associated a plurality of stored images 415, and a moderate zone 416 is associated with a plurality of stored images 417. The images can be stored in one or more databases or memory devices, such as external memory 155 shown in FIG. 1.

A clinician reviewing the images can click or otherwise select an imaging zone of interest on the front view 402 and/or the back view 404 displayed on the GUI 405, and sift through the images corresponding to the selected zone. In this manner, anatomical context is provided for each image being reviewed by the clinician. The clinician can view and/or select certain images for closer analysis, most likely beginning with the images tagged as “moderate” or “severe” by the user who performed the scan. In the illustrated example, image 418 was included within the plurality of images 409 derived from the severe imaging zone 408, and image 420 was included within the plurality of images 417 derived from moderate zone 416. With more time to review, the clinician can agree or disagree with the ultrasound operator's initial severity level estimation, and update the severity status of the images accordingly.

To initiate an image review, a clinician can use the GUI 405 to enter patient-specific information, such as the patient medical record number (MRN), and the system (e.g., system 100) can automatically retrieve all past examination results performed on the patient (e.g., from external memory 155), including results obtained from ultrasound, CT, X-ray, and/or MRI exams. Accordingly, data from one or more non-ultrasound modalities 422 can be communicatively coupled with the ultrasound-based systems described herein. The information from such modalities 422 can also be displayed to the user, for example on GUI 405. In the embodiment represented in FIG. 4, the GUI 405 can display a plurality of CT images 424 and/or X-ray images 426 concurrently with one or more ultrasound images acquired from a particular imaging zone. This consolidation thus allows a clinician to review images obtained from a variety of imaging modalities, each image corresponding to a specific imaging zone.

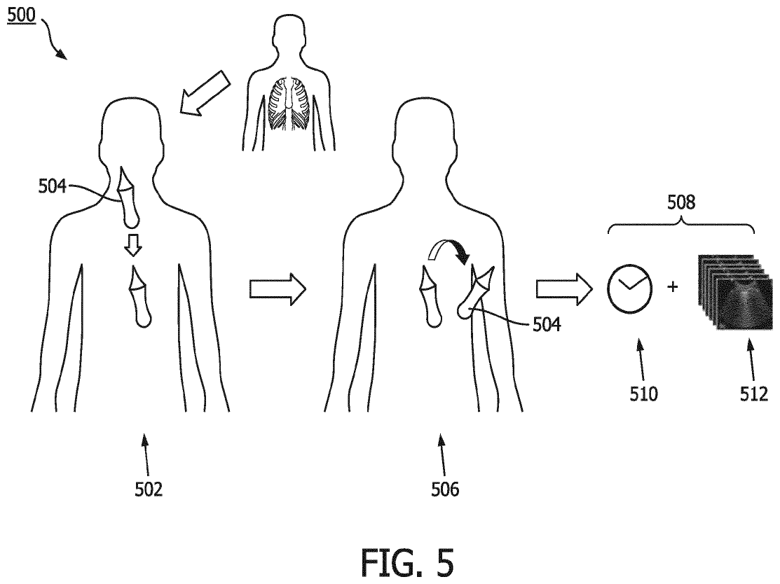

FIG. 5 is a schematic of an ultrasound probe tracking technique 500 implemented according to embodiments described herein. The probe tracking technique 500 may be performed (e.g., via probe tracking processor 150) by utilizing a combination of image data acquired using an ultrasound probe and associated processing components (e.g., probe 112 and image generation processors 134) and motion data acquired using an IMU sensor (e.g., IMU sensor 116). As shown at step 502, a user may translate an ultrasound probe 504 in an inferior direction (represented by the downward arrow) from a superior-most location. A series of ultrasound images can be acquired during this probe movement, which can be used to count or observe anatomical and/or image features, such as ribs. This information can provide a marker to determine the current superior-inferior (S-I) coordinates of the probe. From the determined S-I probe coordinates, the user can tilt and/or slide the probe 504 pursuant to step 506 in a lateral direction until the probe is positioned over an intended image zone. This lateral motion can be tracked with the IMU sensor 116 to derive the lateral/medial and anterior-poster (A-P) probe position.

The probe position determined via the combination of image data and motion data can be augmented by one or more additional factors 508 to determine whether each zone is sufficiently imaged. Non-limiting examples of such factors 508 may include the time spent 510 imaging a particular image zone, the number of ultrasound images 512 acquired at a particular zone, and/or any anatomical or image features recognized within a particular image zone. In various examples, the time spent at a given imaging zone may vary, ranging from less than 30 seconds to about 30 seconds or longer, such as about 2 minutes. The number of images acquired at each zone may also vary, ranging from less than about 5 images to about 5 images, or about 10 images, 15 images, 20 images, or more. Features recognized by the system (for example via feature recognition processor 148) can include the presence of the liver or a portion thereof, the presence of one or more ribs or a portion thereof, and/or the presence of the heart or a portion thereof. The features can also include a variety of abnormalities, such as regions of lung consolidation, pleural lines, and/or excessive B-lines. An abnormality may also be patient-specific, such as a permanent lesion identified during a previous examination. Each of these features may further orient the one or more processors tracking the position of the ultrasound probe (e.g., processor 150), for example by confirming that an imaging zone containing one or more features is currently being imaged. The presence of such features may cause the user to spend more time imaging the zone in which they appear.

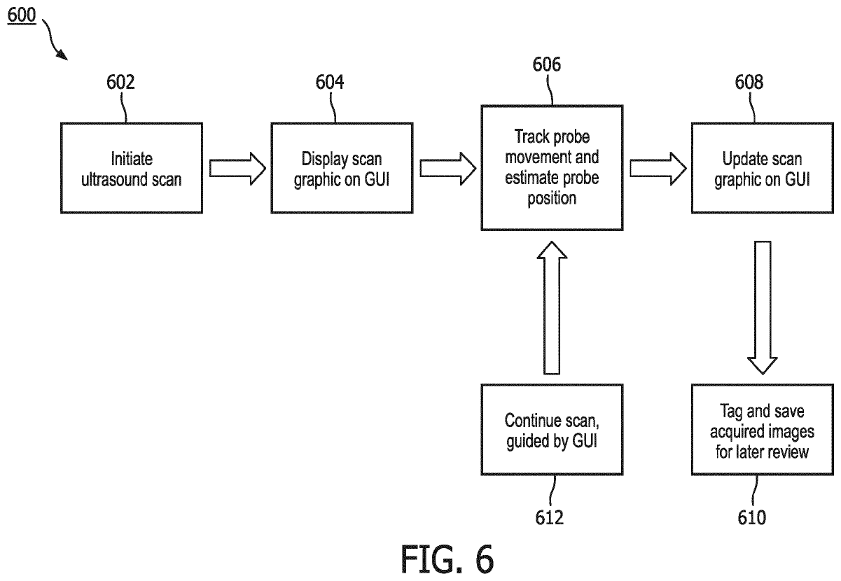

FIG. 6 shows an example method 600 of ultrasound imaging performed in accordance with embodiments described herein. As shown, the method 600 may begin at step 602 by initiating an ultrasound scan with an ultrasound imaging system (e.g., system 100). Initiating the ultrasound scan can involve inputting patient history information, which may involve receiving inputs from the user performing the scan at a graphical user interface (e.g., GUI 124), retrieving patient data from one or more databases (e.g., external memory 155), or both. In some examples, face, voice and/or fingerprint recognition can be used to identify the patient automatically, especially if the patient had a prior scan performed by the same medical institution or department. After identifying the patient, the ultrasound system may retrieve, display and/or implement scan parameters utilized previously to examine the same patient. Such parameters may include the patient's position during the prior scan(s) and/or the particular transducer used. Imaging settings can also be set to match the settings utilized in the previous scan(s). Such settings can include imaging depth, imaging mode, harmonics, focal depth, etc. Initiating the scan can also involve selecting a particular scan protocol, such as a 12-zone protocol used to scan the patient's lungs.

The method 600 can then involve, at step 604, displaying a scan graphic on a GUI viewed by the user. The scan graphic can include one or more imaging zones overlaying a patient graphic, such as shown in FIG. 3, along with the imaging status of each zone. At step 606, the method can involve tracking movement of the ultrasound probe being used and estimating the position of the probe. At step 608, the scan graphic can be updated on the GUI to reflect movement of the probe, along with the time spent at one or more imaging zones and/or the number of images acquired at such zone(s). Step 610 can involve tagging and saving the acquired images for later review. Tagging may involve spatial tagging to associate each image with a particular imaging zone, and/or severity tagging to associate each image with an estimated severity level of a medical condition. At step 612, the method 600 may involve continuing the scan with guidance provided by the updated GUI. Steps 606-612 can then be repeated as many times as necessary to adequately image each imaging zone defined by a particular scan protocol.

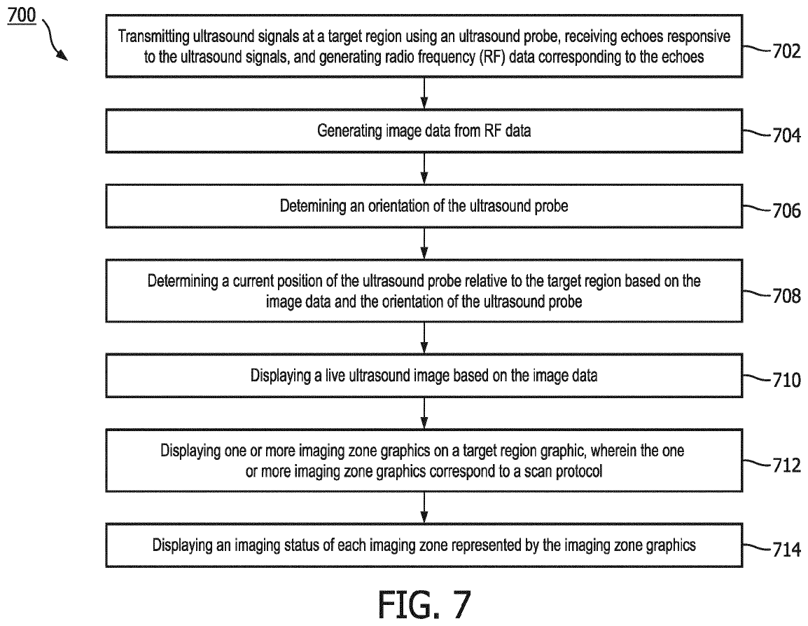

FIG. 7 is a flow chart of an example method 700 implemented in accordance with various embodiments described herein. The method 700 may be performed by an ultrasound imaging system, such as ultrasound imaging system 100. The steps of the method 700 may be performed chronologically in the order depicted, or in any order. One or more steps may be repeated as an ultrasound scan is performed.

At block 702, the method 700 involves transmitting ultrasound signals at a target region (e.g., the lungs of a patient) using an ultrasound probe (e.g., probe 112). Echoes responsive to the signals are then received and RF data is generated therefrom. At step 704, the method 700 involves generating image data from the RF data. This step may be performed by one or more of the image generation processors 134 of system 100. At step 706, the method 700 involves determining an orientation of the ultrasound probe, for example using data obtained by the IMU sensor 116. At step 708, a current position of the ultrasound probe relative to the target region can be determined, for example by the probe tracking processor 150, based on the image data and the orientation of the probe. At step 710, the method 700 may involve displaying, for example on GUI 124, a live ultrasound image based on the image data. Step 712 may involve displaying one or more imaging zone graphics on a target region graphic, for example as shown on the GUI 300 depicted in FIG. 3. The imaging zone graphics can be specific to a scan protocol, for example such that a different number and/or arrangement of graphics may appear depending on the protocol selected by a user. At step 714, the method 700 may involve displaying an imaging status of each imaging zone represented by the imaging zone graphics. The imaging zone status may indicate whether a particular imaging zone has been sufficiently imaged, has yet to be sufficiently imaged, or is in the process of being sufficiently imaged.

In various examples where components, systems and/or methods are implemented using a programmable device, such as a computer-based system or programmable logic, it should be appreciated that the above-described systems and methods can be implemented using any of various known or later developed programming languages, such as “C”, “C++”, “FORTRAN”, “Pascal”, “VHDL” and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software, and/or firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the present disclosure. The functionality of one or more of the processors described herein may be incorporated into a fewer number or a single processing unit (e.g., a CPU) and may be implemented using application specific integrated circuits (ASICs) or general purpose processing circuits which are programmed responsive to executable instructions to perform the functions described herein.

Although the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, examples or processes described herein may be combined with one or more other examples, examples and/or processes or be separated and/or performed amongst separate devices or device portions in accordance with the present systems, devices and methods.

Finally, the above-discussion is intended to be merely illustrative of the present systems and methods and should not be construed as limiting the appended claims to any particular example or group of examples. Thus, while the present system has been described in particular detail with reference to exemplary examples, it should also be appreciated that numerous modifications and alternative examples may be devised by those having ordinary skill in the art without departing from the broader and intended spirit and scope of the present systems and methods as set forth in the claims that follow. Accordingly, the specification and drawings are to be regarded in an illustrative manner and are not intended to limit the scope of the appended claims.

1. An ultrasound imaging system comprising:
an ultrasound probe configured to transmit ultrasound signals at a target region and receive echoes responsive to the ultrasound signals and generate radio frequency (RF) data corresponding to the echoes;
one or more image generation processors configured to generate image data from the RF data;
an inertial measurement unit sensor configured to determine an orientation of the ultrasound probe;
a probe tracking processor configured to determine a current position of the ultrasound probe relative to the target region based on the image data and the orientation of the probe; and
a user interface configured to display:
a live ultrasound image based on the image data;
one or more imaging zone graphics overlaid on a target region graphic, wherein the one or more imaging zone graphics correspond to a scan protocol; and
an imaging status of each imaging zone represented by the imaging zone graphics.

2. The ultrasound imaging system of claim 1, further comprising a graphics processor configured to associate the current position of the ultrasound probe with one of the imaging zone graphics.

3. The ultrasound imaging system of claim 1, wherein the imaging status indicates whether each imaging zone represented by one of the imaging zone graphics has been imaged, is currently being imaged, or has yet to be imaged.

4. The ultrasound imaging system of claim 1, wherein the user interface is further configured to receive a user input tagging at least one of the imaging zone graphics with a severity level.

5. The ultrasound imaging system of claim 1, further comprising a memory communicatively coupled to the user interface and configured to store at least one ultrasound image corresponding to each of the imaging zones.

6. The ultrasound imaging system of claim 1, wherein the imaging status of each imaging zone is based on the current position of the ultrasound probe, a previous position of the ultrasound probe, a time spent by the probe at the current position and the previous position, a number of ultrasound images obtained at the current position and the previous position, or a combination thereof.

7. The ultrasound imaging system of claim 1, wherein the probe tracking processor is configured identify a reference point within the target region based on the image data.

8. The ultrasound imaging system of claim 7, wherein the reference point comprises a rib number.

9. The ultrasound imaging system of claim 7, wherein the probe tracking processor is configured to determine superior-inferior coordinates of the probe based on the reference point.

10. The ultrasound imaging system of claim 9, wherein the probe tracking processor is further configured to determine lateral coordinates of the probe based on the orientation of the probe.

11. The ultrasound imaging system of claim 1, wherein the user interface (124) is further configured to receive a target region selection, a patient orientation, or both.

12. A method comprising:
transmitting ultrasound signals at a target region using an ultrasound probe, receiving echoes responsive to the ultrasound signals, and generating radio frequency (RF) data corresponding to the echoes;
generating image data from the RF data;
determining an orientation of the ultrasound probe;
determining a current position of the ultrasound probe relative to the target region based on the image data and the orientation of the ultrasound probe;
displaying a live ultrasound image based on the image data;
displaying one or more imaging zone graphics on a target region graphic, wherein the one or more imaging zone graphics correspond to a scan protocol; and
displaying an imaging status of each imaging zone represented by the imaging zone graphics.

13. The method of claim 12, further comprising associating the current position of the ultrasound probe with one of the imaging zone graphics.

14. The method of claim 12, wherein the imaging status indicates whether each imaging zone represented by one of the imaging zone graphics has been imaged, is currently being imaged, or has yet to be imaged.

15. The method of claim 12, further comprising receiving a user input tagging at least one of the imaging zone graphics with a severity level.

16. The method of claim 12, further comprising storing at least one ultrasound image corresponding to each of the imaging zones.

17. The method of claim 16, further wherein storing comprises spatially tagging the at least one ultrasound image with the corresponding imaging zone.

18. The method of claim 12, wherein the imaging status of each imaging zone is based on the current position of the ultrasound probe, a previous position of the ultrasound probe, a time spent by the probe at the current position and the previous position, a number of ultrasound images obtained at the current position and the previous position, or a combination thereof.

19. The method of claim 12, further comprising:
identifying a reference point within the target region based on the image data;
determining superior-inferior coordinates of the probe based on the reference point; and
determining lateral coordinates of the probe based on the orientation of the probe.

20. A non-transitory computer-readable medium comprising executable instructions, which when executed cause a processor to:
displaying a live ultrasound image based on the image data;
displaying one or more imaging zone graphics on a target region graphic, wherein
the one or more imaging zone graphics correspond to a scan protocol; and
displaying an imaging status of each imaging zone represented by the imaging zone graphics.
